SEQUENCE LISTING (3 sequences)

SEQ ID NO: 1 - protein, 7 aa, Artificial
  Description of Artificial Sequence Synthetic Cathepsin site
AFRSAAQ

SEQ ID NO: 2 - DNA, 15 bp, Artificial
  Description of Artificial Sequence Synthetic Oligonucleotide for PNA437 and PNA434 binding
caatcggaacggatc

SEQ ID NO: 3 - DNA, 45 bp, Artificial
  Description of Artificial Sequence Synthetic Oligonucleotide for PNA539 and PNA434 binding
gccgaggtgcagcctcaatcggaacggatcggaagggccgagcgc

(19) United States
(12) Patent Application Publication
(10) Pub. No.: US 2005/0260587 A1
(43) Pub. Date: Nov. 24, 2005

(54) Synthetic transfer complex and method for transferring nucleic acids

(76) Inventors: Lars Branden, New York, NY (US); Edvard Smith, Stockholm (SE)

Correspondence Address: YOUNG & THOMPSON, 745 SOUTH 23RD STREET, 2ND FLOOR, ARLINGTON, VA, 22202, US

(21) Appl. No.: 10/512,306

(22) PCT Filed: Apr. 23, 2003
(86) PCT No.: PCT/SE03/00657

§ 371 Date: Jun. 6, 2005

(30) Foreign Application Priority Data
Apr. 24, 2002 (SE) 0201234-2

(51) Int. Cl. C12Q 1/68

(52) U.S. Cl. 435/6

(57) ABSTRACT

A synthetic transport entity is used for transferring a nucleic acid sequence of interest across a biological membrane. The transport entity comprises a functional element (FE), which increases the efficiency of transfection (e.g. polyethylene glycol (PEG) or a nuclear localisation signal), a binding element (BE), such as a peptide nucleic acid (PNA), and a carrier molecule comprising the nucleic acid of interest and a BE target sequence. The transport entity can be altered in a controlled manner at the surface of the membrane or after having passed across the membrane or having been taken up by the cell, and comprises at least one alteration site that, when altered, changes a property of the transport entity.

The present invention relates to the field of biomolecular chemistry and genetic engineering, and in particular to synthetic biomolecular complexes used for transferring sequence information, e.g. in the form of nucleic acids, across a biological membrane, and/or directing it to a specific location in the cell. The present invention in particular concerns complexes having an added functionality with respect to location and/or time, one or more properties that change with time in a controlled manner.

### BACKGROUND OF THE INVENTION

Gene transfer can be defined as the cellular uptake of nucleic acids. The efficiency of DNA transfection is dependent on several steps: adsorption of the transfection complex to the cellular surface, uptake by the cell, escape from the endosome/lysosome, nuclear translocation with or without chromosomal integration, and the effect of the nucleic acid. Cellular uptake of nucleic acids and expression of the nucleic acid as proteins are the basis of gene therapy. Gene therapy is used as an approach to treat, alleviate, cure, or ultimately prevent a disease by changing the expression of a gene. Inherited genetic diseases, cancer, cardiovascular diseases, autoimmune diseases, AIDS, and neurodegenerative diseases are examples of biological disorders for which research is striving to develop gene therapy methods as treatment.

A gene delivery tool, gene carrier (vector), is usually necessary in order to carry out gene transfer. Commonly used vectors for genetic transfer are viruses. Viruses have evolved a way of encapsulating and delivering their genes to cells in a pathogenic manner. By taking advantage of the biology of the viruses while manipulating their genomes to remove the disease-causing genes, therapeutic genes can be inserted into the cell genome. The most commonly used viruses are from the retroviridae- and the adenoviridaefamilies. The utilization of viruses is an effective way to introduce genetic material into a cell. However, there is a risk that viruses might introduce other problems to the body, such as toxicity, immune and inflammatory responses. Another limitation with viral vectors is their difficulty in targeting to specific cell types.

Alternative vectors based on synthetic, non-viral systems have been developed. The simplest non-viral system is the use of naked expression vector DNA (plasmid DNA). Other examples of non-viral DNA transfer are liposomes and molecular conjugates. Although direct injection of free DNA has been shown to lead to gene expression the overall expression is lower than with either viral or liposomal vectors. Liposomes are lipid bilayers encapsulating a fraction of aqueous fluid. DNA will spontaneously associate to the external surface of cationic liposomes and these liposome/DNA complexes will interact with the cell membrane. Compared to viral vectors, the transfection efficiency of liposome/DNA complexes in vivo is relatively low. Molecular conjugates consist of naturally occurring or synthetic ligands and are used as gene delivery systems.

Standard, non-viral, methods of transfection suffer from the inefficient nuclear uptake of the genetic material introduced into the cell. Improving the non-viral gene delivery systems is therefore of great relevance. Further increase of the in vivo expression of transferred DNA by efficient and specific transport into the cell would of course confer improvements to gene therapy technology.

### PRIOR ART

WO 99/13719 presents a method for chemical modification of DNA using peptide nucleic acid (PNA) conjugates and an approach to permanently introduce new physical and biological properties into DNA. A nucleic acid molecule and a PNA molecule are associated with a DNA molecule. The PNA molecule contains a region complementary to the DNA molecule forming a so called PNA clamp. The PNA can be labelled (e.g. fluorescently or enzymatically) for determination of the biodistribution of exogenous transfected nucleic acid molecules in a cell. In addition the PNA (or PNA clamp) may be conjugated to a protein, peptide, carbohydrate moiety or receptor ligand and the formed complexes are used to increase the efficiency of expression of a particular gene.

WO 00/15824 also discloses a method for transfer of a nucleic acid across a biological membrane and specific localization of said nucleic acid within a cell. The method is based on the use of a synthetic transport entity composed of a functional element and a binding element, preferably separated by a linker molecule. The functional element can be, for example, a nuclear localization signal (NLS) that confers a specific biological function to a molecule linked to it. PNAs are used as binding elements, which hybridize to a specified target present on a carrier of the nucleic acid of interest. In contact with the biological membrane the transport entity will provide for a transfer of the nucleic acid of interest across the biological membrane.

The present invention aims to improve the above and similar methods with respect to safety, functionality, efficiency and stability, i.e. to set aside possible unwanted effects due to non-sequence portions of the transport entity or biomolecular complex interfering with the normal functions of the cell or with the expression of the sequence. Another aim is to make it possible to alter properties of the complex, for example to control the activation and/or deactivation of certain functions of the transfer entity or biomolecular complex within the cell. Yet another aim is to make it possible to use shorter sequences with retained specificity and stability.

### SUMMARY OF THE INVENTION

The present invention presents a synthetic transport entity for transferring a nucleic acid sequence of interest across a biological membrane, and/or direction thereof to a specific location within a cell. The transport entity comprises at least one functional element (FE), a binding element (BE), and a carrier molecule comprising the nucleic acid of interest and a target sequence complementary to the BE. The transport entity can be altered in a controlled manner prior to the use in biological system, at the surface of the membrane or after having passed across the membrane or having been taken up by the cell, and comprises at least one alteration site that, when altered, changes a property of the transport entity. The present invention also relates to a method for transfer the transport entity across a biological membrane, and/or direction thereof to a specific location within a cell as well as a kit for making the transport entity.

### DESCRIPTION OF THE FIGURES

The invention will be disclosed in further detail in the following description, examples and attached drawings, in which

FIG. 1 shows schematically an embodiment of the present invention. When the linkers are cleaved, the separate parts of the binding element (BE) are too short to obtain a stable hybridization and thus, the transport entity, including the functional element (FE), will dissociate from the target sequence.

FIG. 2 shows schematically an embodiment of the present invention. When the PNA/DNA complex is in a helical shape the length of the linking molecule is suitable to bind to the opposite ends of the PNA sequence through disulfide bonding, thus stabilizing the PNA/DNA association. FE2 and FE3 denote two functional units, which can be attached to each other via binding, for example by disulfide linkage following oxidation or by linkage induced by chemical crosslinkers.

FIG. 3 is a graphical presentation of controlled release by enzyme cleavage in a PAGE retardation assay. Comparison of in vitro and in vivo digestion of bioplex with exogenous or endogenous cathepsin-L enzyme, respectively (bioplex is here used as a name for an oligonucleotide hybridized to a BE-FE complex carrying a cathepsin cleavage site in the linker between the BE and the FE).

FIG. 4 shows the results from the visualization of disulfide linked PNA-peptides by hybridization to an oligonucleotide. The oligonucleotide is hybridized with PNA539 under oxidized and reduced conditions. Lane 1—free oligo; Lane 2—hybridized at oligo: PNA ratio 1:1 reduced conditions; Lane 3-hybridized at oligo: PNA ratio 4:1 oxidized conditions; Lane 4—hybridized at oligo: PNA ratio 1:1 oxidized conditions; Lane 5—hybridized at oligo: PNA ratio 1:2 oxidized conditions; A-Two oligos and two PNAs connected with a disulfide bridge; B-One oligo with two PNA connected with a disulfide bridge; C-Oligo with one PNA; E-Free oligo.

FIG. 5 shows the results from the hybridization of PNA to a binding element sequence of varying length. The number of bases in the PNA″ refers to the number of bases being anti-sense to the oligonucleotide and its mutated derivatives.

FIG. 6 shows chemical crosslinking of two different FEs. A-DNA/PNA at ratio of 1:1, no reduction; B-DNA/PNA434 at ratio of 2:1, hybridized and reduced with 2 mM DTT followed by the addition of the chemical crosslinker; w-position of unhybridized oligonucleotide; x-position of DNA/PNA434 crosslinked with PNA542; y-position of DNA/PNA434; z-position of DNA/PNA434 disulfide bound to a second PNA434.

### DETAILED DESCRIPTION OF THE INVENTION

In the present description and claims, the following terms and abbreviations will be used:

The term “functional element” (FE) is used to denote any moiety capable of conferring one or more specific properties and/or biological functions to a molecule linked to it.

A “binding element” (BE) may be any natural or synthetic nucleic acid, nucleic acid derivative or nucleic acid analogue capable of specific, strong and durable binding to a specified target thereof, preferably by hybridization. One example of such BE is the PNA described below.

A “target” or “target region” is a specific region corresponding to a BE, and may be any natural or synthetic nucleic acid, nucleic acid derivative or nucleic acid analogue capable of specific, strong and durable binding to a specified BE, preferably by hybridization.

A “carrier molecule” may be a plasmid, a vector, an oligonucleotide or any other molecule or construct capable of harboring and/or transferring nucleic acids during genetic modification events.

A “linker” (L) may be any chemical structure connecting two BEs, a FE and a BE, or two opposite ends of a BE, defining a distance and orientation between these. Preferably the linker does not participate in the chemical/biochemical interactions of the FEs. The linker is preferably a natural or synthetic nucleic acid polymer, but may also be any suitable synthetic or natural polymer.

The term “alteration site” is used to define a location in the transport entity, capable of undergoing a change which results in an altered property of the transport entity, such as an altered chemical property, functional property, structural or spatial property, as compared to the corresponding property of said transport entity prior to said change. An altered property may also be the dissociation or restructuring of the components of the transport entity. Such “alteration site” may be a restriction site or recognition site, cleavable by specific enzymes, or a chemical bond, cleaved enzymatically or chemically, and/or at a specific conditions, such as reduction, oxidation, change in temperature or in the presence of particular reactants. Said “alteration site” may also comprise several bonds, such as the bonds between one end of a part of the transport entity, a linker, and another end of said transport entity.

“PNA” is an acronym for Peptide Nucleic Acid, which is a DNA mimic having a pseudopeptide backbone consisting of aminoethyl glycine units, to which the nucleobases are attached via methylen carbonyl linkers. A PNA molecule is capable of hybridizing to complementary ssDNA, dsDNA, RNA and PNA targets. The neutral backbone of PNA, in contrast to the negatively charged DNA, results in strong binding and high specificity. In the present application, it is to be understood that the term “PNA” refers to any DNA analogue comprising the above backbone and nucleobases, and the term is thus not limited to the specific structures disclosed herein.

A “label” or “marker” is a composition detectable by spectroscopic, photochemical, biochemical, immunological or chemical means.

“Cathepsins” are enzymes that are members of the papain family of cysteine proteinases and are involved in many normal cellular processes and a number of pathologic conditions. Cathepsins represents a major component of the lysosomal proteolytic system and are expressed in many different cell types.

The present invention makes available a synthetic transport entity for transferring a nucleic acid sequence of interest across a biological membrane, and/or direction thereof to a specific location within a cell. The transport entity comprises at least one functional element (FE), a binding element (BE), and a carrier molecule comprising the nucleic acid of interest and a target sequence complementary to the BE. Importantly, the transport entity can be altered in a controlled manner at the surface of the membrane or after having passed across the membrane or having been taken up by the cell, and comprises at least one alteration site that, when altered, changes a property of the transport entity. The transport entity may also comprise more than one FE, which can be the same or different.

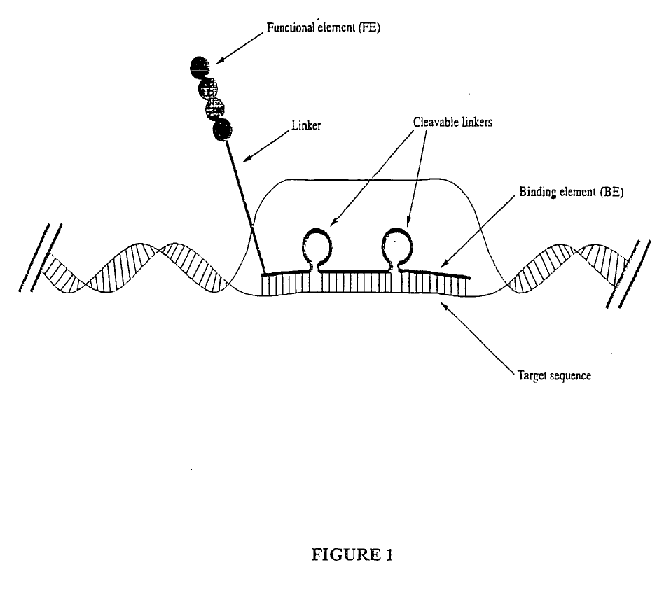

In one embodiment of the present invention one alteration site is located between the BE and FE. If this alteration site is cleaved, only the FE will dissociate. In another embodiment the alteration site/-s is/are located between two or more parts of the BE. If the alteration site/-s is/are cleaved, the separate parts are constructed so that they will be too short to hybridize to the BE target sequence after cleavage and thus, both the FE and BE will dissociate from the carrier molecule (FIG. 1). Preferably the alteration site/-s is/are cleaved enzymatically by surface bound proteases and/or intracellular proteases, such as granulocyte serine proteases, ATP-dependent mitochondrial proteases or cytosolic cysteine proteases. More preferably, when the enzyme is an intracellular protease, the alteration site/-s is/are cleaved by cathepsin-like enzymes at site/-s comprising a predefined sequence. In the specific case when cathepsins are used, the alteration site/-s is/are:

| | |
|---|---|
| afrsaaq | (SEQ. ID. NO. 1) |

In addition, the synthetic transport entity may further comprise at least one alteration site in the form of a disulfide bond.

The transport entity of the invention may include a marker or a label, such as a fluorescent label, a chemical label, a radioactive label etc., to enable detection and identification of the cells that have included the entity.

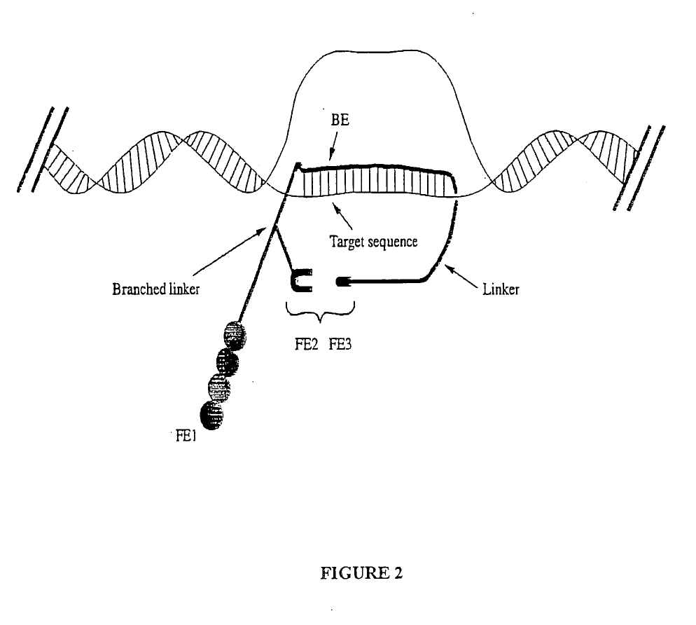

In yet another embodiment one alteration site is located between FE and BE and one end of the BE sequence is coupled to the opposite end of the BE sequence by a linking sequence through disulfide bonds or a chemical crosslinker, causing a stabilization of the transport entity (FIG. 2). The stabilization can occur prior to the use in biological system, but after the hybridization between the BE and the transport entity, or after transfection, at the surface of the membrane or after having passed across the membrane or having been taken up by the cell. When the carrier molecule is helically shaped, the length of the linking molecule is appropriate and the linkage at the two ends of the BE sequence becomes possible. The BE sequence is a sequence comprising a number of nucleotides sufficient to obtain stable association, the number of nucleotides being less than would be possible in a method not comprising the coupling linker sequence. In the specific case when BE is a PNA molecule a low number of nucleobases (15 nucleobases or less) is preferable, provided the stabilization of the PNA/DNA complex is not negatively affected. Under physiological conditions, which would normally require a higher number of nucleobases to retain a stable association, a low number of nucleobases is feasible due to the coupling linker sequence maintaining the stable association. By changing the conditions for the association the number of nucleobases can be minimized without reducing the stability of the PNA/DNA complex.

In a preferred embodiment of the transport entity relating to stabilization of the BE sequence, the transport entity is adapted to dissociate at the surface of the biological membrane or after having passed across the biological membrane or having been taken up by the cell by altering at the alteration site site present between the FE and BE. Preferably the alteration site is cleaved enzymatically by surface bound proteases or intracellular proteases, such as granulocyte serine proteases, ATP-dependent mitochondrial proteases or cytosolic cysteine proteases. More preferably, when the enzyme is an intracellular protease, the recognition site it is cleaved by cathepsin-like enzymes at a site comprising a predefined sequence. In the specific case when cathepsins are used, the recognition site is SEQ ID NO 1. The alteration site can also comprise disulfide bonds.

More functions can be linked to the complex to increase the transfection efficiency of the transport entity. Candidate molecules are for example: RGD (receptor mediated endocytosis), HA2 (lysosomal escape), sugar moieties (receptor mediated endocytos in hepatocytes), and transferrin receptor ligand (receptor mediated endocytosis). These can be linked either to BEs with different recognition sites or linked to the same BE, function after function. The possibility to eliminate a functional moiety after it has served its purpose is advantageous.

In the specific case when the BE is a PNA molecule and multiple functions are located on the same PNA in a sequential manner it is important to be able to cleave of the terminal peptide that otherwise would shield the inner peptide. By adding a specific recognition site between the functions and/or between the PNA and the function, elimination of functional moieties can be achieved. The alteration site, e.g. a protease site can be chosen in such a way that the function is cleaved off as a result of the change of environment that the transfection complex has been targeted to.

In a specific embodiment when the transport entity is used to shield the nucleic acid of interest, for example at an intravenous injection, the FE is a suitable polymer e.g. polyethylene glycol (PEG) and the BE is PNA.

The present invention also relates to a method for transferring a nucleic acid sequence of interest across a biological membrane, and/or direction thereof to a specific location within a cell, wherein a synthetic transport entity defined above is used for transfection.

The present invention also relates to a cell transfected with the synthetic transport entity defined above. Consequently, the invention also relates to such cell therapy methods as well as to cells used therein that have been genetically modified by a transfer entity and/or a method according to the present invention.

The present invention also relates to a kit comprising components for making a transport entity capable of transferring a nucleic acid sequence of interest across a biological membrane, and/or direction thereof to a specific location within a cell and components for altering said transport entity in a controlled manner. The kit comprises at least one FE, a binding element (BE), and a carrier molecule comprising the nucleic acid of interest and a target sequence complementary to the BE. The components in the kit comprise at least one alteration site.

### EXAMPLES

### Example 1

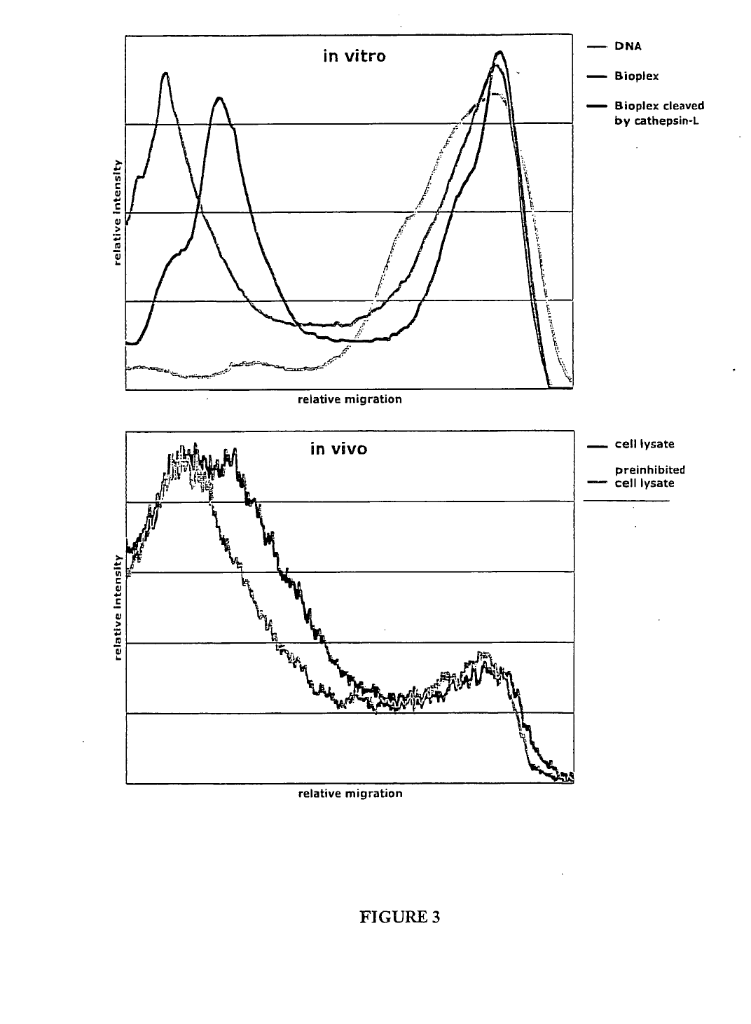

### (FIG. 3) Controlled Release of Peptide Moities from PNA/DNA Transfection Complexes

Cathepsin-L was used to cleave a PNA-FE complex with a linker containing the cathepsin-L site arfsaaq. Cathepsin-L is ubiquitously expressed in eukaryotic cells and located in the endosomal vesicles. It becomes active as the pH drops when the early endosome matures into a late endosome/early lysosome. The PNA in the experiment was bi-functional and consisted of a nucleic acid binding element and RGD as a functional entity to enhance endosomal uptake.

Material and Methods:

Cell Line and Medium

NIH 3T3 cells (embryonic mouse fibroblast) cultivated in DMEM with 10% fetal calf serum supplemented with 100 μg/ml gentamicin.

PNA437 and DNA Oligonucleotide

PNA was synthesised on a Expedite 8909 or equivalent machine. Oligonucleotides have been synthezised on an Applied Biosystems International DNA syntheziser. Linker(L)-peptide, upper case letters denote the PNA bases, whereas lower case letters denote amino acids in the FE.

PNA437:

N-term GAT CCG TTC CGA TTG-LafrsaaqLLcyggrgdtp
C-term

DNA Oligonucleotide for PNA437 Binding:

| | |
|---|---|
| 5′- CAA TCG GAA CGG ATC -3′ | (SEQ. ID. NO. 2) |

Labeling

T4 poly nucleotide kinase was used to label one end of the target oligonucleotide with γ³²-ATP according to the protocol supplied by the manufacturer (Promega).

Hybridization

Hybridization between PNA437 and DNA-oligonucleotide was performed at 95° C. for 3 minutes and room temperature for 2 hours in water with a two-fold excess of PNA437. The total hybridization volume was 10 μl.

In vivo

50,000 NIH 3T3 cells in each well of a 24-well dish were transfected with PNA-hybridized oligonucleotide at 37° C. in a CO₂-incubator. The transfection time was 6 h and 15 pmol PNA-hybridized oligonucleotide per well was used. The abundance of PNA/oligonucleotide complex ensures sufficient transfection. The experiment was performed in duplicate and as a negative control a sample treated with a cathepsin inhibitor (a cell permeable and irreversible cathepsin-L inhibitor, final concentration 15 μM) was used. To remove unabsorbed PNA/oligonucleotide complex the cells were washed 3 times with PBS at 4° C. Cell lysis was accomplished by treatment with H₂O. The cell lysates were analyzed on 15-% non-denaturing PAGE (poly acrylamide gel electrophoresis) for 3h at 90V and imaged on a BioRad Molecular Imager FX pro plus phosphorimager.

In vitro

PNA437 with a cathepsin-L site was hybridized to the DNA oligonucleotide as previously described and the PNA/DNA complex was subsequently incubated for 1h at room temperature with cathepsin-L (at a concentration recommended by the manufacturer; Calbiochem, US) in cathepsin-L reaction buffer (supplied from the manufacturer) for 1, 15, and 30 minutes. PNA/DNA complex not treated with cathepsin-L and free oligonucleotide was used as control. The different reactions were separated on a 15-% non-denaturing PAGE for 3h at 90V and imaged on a BioRad Molecular Imager FX pro plus phosphorimager.

Results

In vivo

The results showed that peptide moieties could be cleaved from the transfected molecular complexes via sequence specific recognition of cathepsin-L (FIG. 3, lower graph). Furthermore, the cathepsin-L activity was blocked with a specific cathepsin-L inhibitor. The in vivo study was compared with in vitro digestion of an aliquot of the hybrids (performed as described previously for in vitro studies). Digestion was seen both in vivo and in vitro, although complete digestion was not obtained in the in vivo experiment.

In vitro

The in vitro results show that the PNA/DNA complex can carry a temporary biological function and after sequence specific protease digestion part of the transfection complex can be removed (FIG. 3). The cleavage reduced the size of the complex. When the cleavage is complete a single band appears, but when it is incomplete two bands appear. In this experiment the cleavage was complete after 30 minutes.

### Example 2

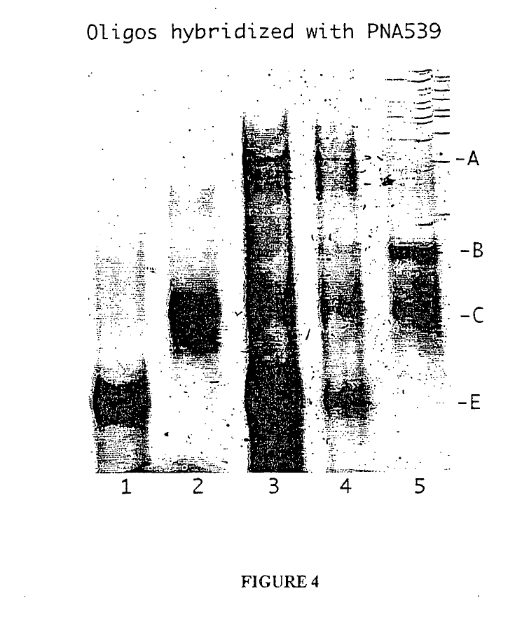

### (FIG. 4) Visualization of Disulfide Linked PNA-Peptides By Means of Hybridization to Oligonucleotides

In this experiment an oligonucleotide was hybridized with a cysteine-containing PNA-peptide under oxidizing and reducing conditions to show that disulfide bridges will not interfere with the formation of PNA-peptide-DNA complexes, and that it is possible to connect and release two DNA molecules from each other via the disulfide bridge formed between the hybridized PNA-peptides.

Material and Methods

PNA-peptide and Oligonucleotide

PNA539:

N-term cggLL-GAT CCG TTC CGA T-LLLggggc C-term

was hybridized to a single binding element (BE) in the center of a 45-mer oligonucleotide with the sequence:

(SEQ ID. NO. 3)

5′-GCC GAG GTG CAG CCT CAA TCG GAA CGG ATC GGA AGG
GCC GAG CGC-3′

with the BE underlined. (SEQ ID. NO. 3)
Hybridization

Hybridizations were performed by heating PNA and oligonucleotides at 95° C. for 3 min, cooling to room temperature and continued incubation for 2 hrs. The hybridizations contained 20 pmol PNA and 10-40 pmol oligonucleotide and (ratios as indicated in figure legend) in 10 mM Na₂PO₄ buffer pH 6.8.

Reduction and Oxidation

PNA was diluted to 10 mM in 10 mM phosphate buffer pH 6.8 and incubated at room temperature over night to allow maximal oxidation of sulfhydryl groups by the air oxygen. Reduction of disulfide bonds was achieved by including 1 mM DTT in the hybridization reaction (FIG. 4 lane 2).

Analysis

The hybridization results were analyzed on a 15% native polyacrylamide gel in 1×TBE (90 mM tris-borate buffer, 2 mM EDTA, pH 8.3). The gel was stained with SYBR-green to visualize the oligonucleotide. Hybridization was indicated by reduced mobility of the oligonucleotide in proportion to the size of the complex.

Results

The results are shown in FIG. 4. When PNA-peptide chimera containing cysteine residues are incubated under oxidizing conditions a fraction of the molecules form disulfide bonds. When hybridized to a DNA molecule such PNA form different complexes in relation to the ratio between the PNA and the oligonucleotide. At high PNA to oligonucleotide ratio (2:1) most oligonucleotides are hybridized with a PNA-peptide connected to another PNA-peptide via a disulfide bridge (lane 5, position B). At lower ratios (lanes 3 and 4) higher molecular weight complexes are formed with two oligonucleotides each hybridized to a PNA-peptide, and the two PNA-peptides connected with a disulfide bridge (position A). Under reducing conditions in presence of 1 mM DTT (lane 2) all disulfide bridges are reduced and only complexes corresponding to one PNA hybridized with one oligonucleotide (position C) can be found. Free non-hybridized oligonucleotide is found in position E.

This experiment shows that PNA-peptide chimera containing cysteine residues can participate in disulfide bridges also when bound to a DNA molecule via PNA-DNA hybridization.

### Example 3

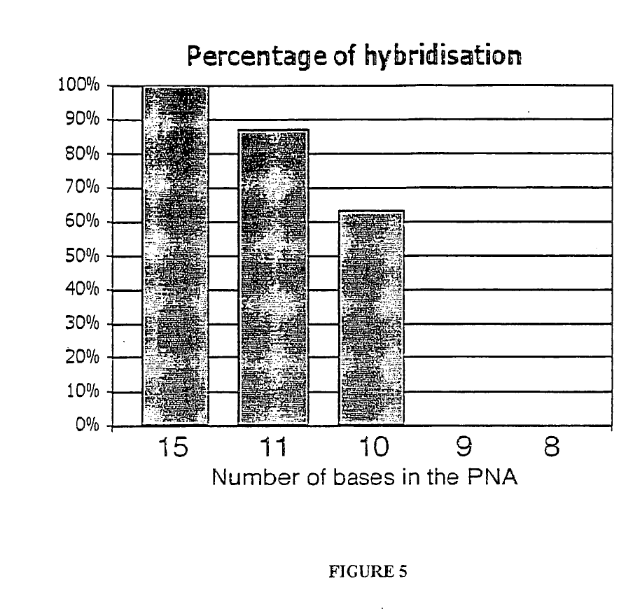

### (FIG. 5) Hybridization of PNA to a Binding Element Sequence of Varying Length

Material and Methods

PNA434:

N-term GAT CCG TTC CGA TTG-LLLcyggrgdtp C-term

was hybridized to a single binding element (BE) in the center of a 45-mer oligonucleotide with the sequence:

(SEQ ID. NO. 3)

5′-GCC GAG GTG CAG CCT CAA TCG GAA CGG ATC GGA AGG
GCC GAG CGC-3′

(the BE underlined).
in an anti-parallel direction. An increasing number of bases were mutated in the 3 ′ end of the BE to create oligonucleotides with BEs of different sizes. The amount of the oligonucleotide that was gel shifted after hybridization at a PNA to DNA ratio of 2:1 is indicated in percent in FIG. 5.

Hybridizations were performed by heating PNA and oligonucleotides at 95° C. for 5 min, cooling to room temperature and continued incubation for at least 2 hrs. The hybridizations contained 10 pmol oligonucleotide and 20 pmol PNA in 10 mM Na₂PO₄ buffer pH 6.8 with a final PNA concentration of 1 μM. The hybridization results were analyzed on a 15% native polyacrylamide gel in 1×TBE (90 mM tris borate buffer, 2 mM EDTA, pH 8.3) and the gel was stained with SYBR-green to visualize the oligonucleotide. The gel was exposed in Fluor-S MultiImager, and the ratio of shifted oligonucleotide was quantified using the Quantity One software.

Results

A slight reduction in binding was observed when the BE was 11 nucleotides as compared to (FIG. 5), whereas binding to BE with 9 or 8 nucleotides was abolished. Similar results were obtained when nucleotides were mutated from the 5′ or 3′ end.

This experiment demonstrates that a short BE will dissociate from the BE target sequence. Two 8-oligomers might for example be coupled through a cathepsin recognition site. When the transport entity enters the endosome, the cathepsin site will be cleaved when the pH decreases leading to dissociation of the transport entity.

### Example 4

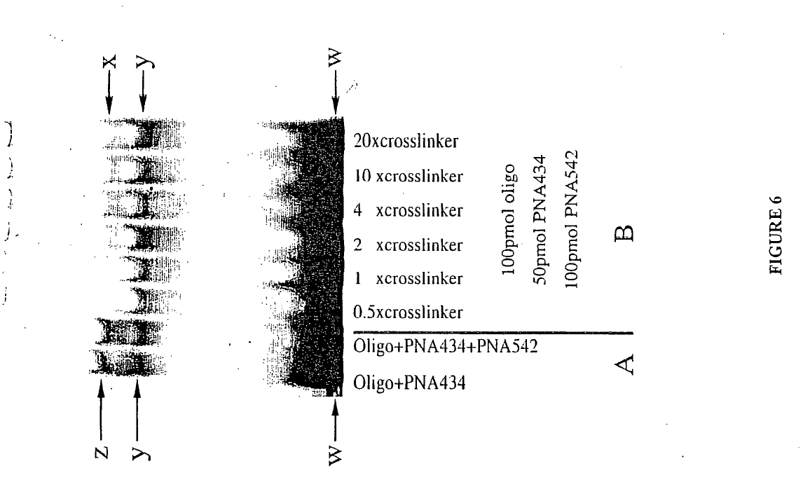

### (FIG. 6) Chemical Cross-Linking Using a Non-Cleavable Linker

Material and Methods

DNA/PNA

PNA434

N-term GAACCTGCTATCTAG-LLLcyggrgdtp C-term

PNA542

N-term-cgACGGATCTCAATCGGgc-C-term

DNA Oligonucleotide for PNA434 Binding:

| | |
|---|---|
| 5′- CAA TCG GAA CGG ATC -3′ | (SEQ. ID. NO. 2) |

Hybridization

Hybridization between DNA and PNA434 was performed at 95° C. for 3 minutes and 2 hours at room temperature. Final concentration of oligonucleotide was 10 pmol/μl diluted in water. The BE sequence in the oligonucleotide is anti-sense to PNA434, whereas the BE of PNA542 has no corresponding sequence for hybridization to this oligonucleotide. Crosslinking PNA434 with PNA542 gave a detectable mass increase of the complex when analyzed by gel retardation assay.

Cross-Linking

A DNA/PNA at ratio of 1: 1, no reduction.

B DNA/PNA434 at ratio of 2:1, hybridized and reduced with 2 mM DTT. Thereafter the crosslinker BM[PEO]3(1,8-bis-Maleimidotriethyleneglycol) an 8-atom polyether spacer for sulfhydryl-to-sulfhydryl cross-linking applications was added. The Reactive groups, maleimide (homobifunctional), are reactive towards sulfhydryl groups. Maleimides react with —SH groups at pH 6.5-7.5, forming stable, non-reversible and non-cleavable thioether linkages. The spacer is water-soluble, and non-cleavable. BM[PEO]3 was added at an excess, as indicated, 30 minutes prior to the addition of 100 pmol PNA542 followed by an additional incubation for 2 hours at room temperature.

Results

This experiment demonstrates the possibility of actively inducing non-cleavable cross-linking, which is the same principle as is used to induce cross-linking between a linking sequence and FE as well as binding of FE to BE according to the invention. Thus, in the lanes “Oligo+PNA 434”, or “Oligo+PNA434+PNA542” the z-band corresponds to the oligo hybridized to PNA434, which is disulfide-bonded to another PNA434, thus forming a PNA434 dimer, whereas the y-band corresponds to the oligo hybridized to non-disulfide-bonded, single PNA434 and the w-band represents free oligo. In panel A, addition of the PNA542 oligo did not cause any changes in mobility.

The reduction performed in the B panel resulted in the loss of the disulfide-bonded complex corresponding to the z-band. Adding crosslinker and subsequently the PNA542 PNA-peptide resulted in a concentration-dependent appearance of a crosslinker (x) x-band formed by PNA542 chemically crosslinked using the BM[PEO]3 reagent to the hybridized PNA434, the z-band being similar to the x-band in size, since the two different PNA-peptides (PNA434 and PNA542) are of similar lengths.

Although the invention has been described with regard to its preferred embodiments, which constitute the best mode presently known to the inventors, it should be understood that various changes and modifications as would be obvious to one having the ordinary skill in this art may be made without departing from the scope of the invention as set forth in the claims appended hereto.

1-25. (canceled)

26. A synthetic transport entity for transferring a nucleic acid sequence of interest across a biological membrane, and/or direction thereof to a specific location within a cell, said transport entity comprising at least one functional element (FE), a binding element (BE), and a carrier molecule comprising the nucleic acid of interest and a target sequence complementary to the BE, characterized in that said transport entity can be altered in a controlled manner at the surface of said membrane or after having passed across said membrane or having been taken up by the cell, said transport entity comprising at least one alteration site that when altered changes a property of said transport entity, said alteration site/-s is/are located between two or more parts of the BE, the separate parts alone being too short to hybridize to the BE target sequence.

27. The synthetic transport entity according to claim 26, wherein said at least one alteration site is a sequence cleavable by an intracellular protease.

28. The synthetic transport entity according to claim 26, wherein said at least one alteration site is a sequence cleavable by a surface bound protease.

29. The synthetic transport entity according to claim 26, wherein said at least one alteration site is a predefined sequence which is cleaved by a cathepsin-type enzyme.

30. The synthetic transport entity according to claim 30, wherein said predefined sequence is the afrsaaq-sequence (SEQ ID NO 1)

31. The synthetic transport entity according to claim 30, wherein the enzyme is cathepsin-L.

32. The synthetic transport entity according to claim 26, wherein said at least one FE is PEG.

33. The synthetic transport entity according to claim 26, further comprising at least one alteration site in the form of a disulfide bond.

34. The synthetic transport entity according to claim 26, wherein said at least one alteration site comprises one end of said BE sequence coupled to the opposite end of the BE sequence by a linking sequence through disulfide bonds.

35. The synthetic transport entity according to claim 26, wherein said at least one alteration site comprises one end of said BE sequence coupled to the opposite end of the BE sequence by a chemical crosslinker.

36. The synthetic transport entity according to claim 34, wherein the coupling of said BE sequence to the opposite end of the BE sequence occurs prior to the use in biological system, but after the hybridization between the BE and the transport entity.

37. The synthetic transport entity according to claim 34, wherein said BE sequence is a sequence comprising a number of nucleotides sufficient to obtain a stable hybridization, the number of nucleotides being less than would be possible in a method not comprising a coupling linker sequence.

38. The synthetic transport entity according to claim 34, wherein said at least one alteration site is cleavable by an intracellular protease.

39. The synthetic transport entity according to claim 34, wherein said at least one alteration site is cleaved by a surface bound protease.

40. The synthetic transport entity according to 34, wherein said at least one alteration site is a predefined sequence which is cleaved by a cathepsin-type enzyme.

41. The synthetic transport entity according to claim 40, wherein said predefined sequence is the afrsaaq-sequence (SEQ ID NO 1).

42. The synthetic transport entity according to claim 40, wherein the enzyme is cathepsin-L.

43. The synthetic transport entity according to claim 34, wherein said at least one FE is PEG.

44. The synthetic transport entity according to claim 34, further comprising at least one alteration site in the form of a disulfide bond.

45. A method for transferring a nucleic acid sequence of interest across a biological membrane, and/or direction thereof to a specific location within a cell, wherein a synthetic transport entity according to claim 26 is used for transfection.

46. A cell transfected with the synthetic transport entity according to claim 26.

47. A kit comprising components for making a transport entity capable of transferring a nucleic acid sequence of interest across a biological membrane, and/or direction thereof to a specific location within a cell and components for altering said transport entity in a controlled manner, said kit comprising at least one FE, a BE, and a carrier molecule comprising the nucleic acid of interest and a target sequence complementary to the BE, and said components comprising at least one alteration site, said alteration site/-s is/are located between two or more parts of the BE, the separate parts alone being too short to hybridize to the BE target sequence.
